(19) United States
(12) Patent Application Publication
(10) Pub. No.: US 2025/0221696 A1
(43) Pub. Date: Jul. 10, 2025

(54) Retraction System for Maxillofacial Surgery

(71) Applicant: Ihsan Tasci, Cankaya-Ankara (TR)

(72) Inventor: Ihsan Tasci, Cankaya-Ankara (TR)

(21) Appl. No.: 18/406,163

(22) Filed: Jan. 7, 2024

(51) Int. Cl. A61B 17/02; A61B 17/24; A61B 90/14

(52) U.S. Cl. CPC A61B 17/0218; A61B 17/24; A61B 90/14

(57) ABSTRACT

Present invention provides a retractor carrier device including two carriers aligned substantially parallel to each other to allow applying of screws and nails into the jaw. Functional joints that can be fixed in the desired angle and shape interconnect the two carriers.

### TECHNICAL FIELD OF THE INVENTION

The invention relates generally to surgical retractor carriers for maintaining an open state of a patient's oral cavity. In particular, the invention relates to a retractor carrier system for maxillofacial surgery.

### BACKGROUND OF THE INVENTION

Retractors used to provide access to a patient's oral cavity during surgical procedures are generally known. Known devices rely at least partially on auxiliary staff to keep the patient's oral cavity open safely and reliably, because of limitations of retractor carrier systems due to problems to adapt to different kind of face types.

During a manual or robotic surgery, an oral retractor carrier device is needed which does not prevent surgeons to reach surgical sites, does not require an auxiliary staff, and allows easy intervention to every point of the chin.

### BRIEF DESCRIPTION OF THE PRESENT INVENTION

Present invention provides an ergonomic retractor carrier device from which any point of the chin may be reached and which may easily adapt to the shape of the chin and face of the patient. The inventive retraction device is in such a way that it cannot prevent the surgeon from working at any point of the chin and it can be applied to one jaw or both jaws at the same time if desired. Fixation structures of the inventive retraction device are arranged outside of the surgeon's working area.

Present invention provides a retractor carrier device including two carriers aligned substantially parallel to each other to allow applying of screws and nails into the jaw. The two carriers are interconnected by functional joints that can be fixed in the desired angle and shape.

The present invention provides an oral retractor carrier device, comprising a longitudinal first rack having a longitudinal first guide portion; a longitudinal second rack having a longitudinal second guide portion; wherein the longitudinal first guide portion and the longitudinal second guide portion are aligned substantially parallel and coupled to each other such that the longitudinal second rack is arranged movably with respect to the longitudinal first rack. The oral retractor carrier device may further comprise at least a connector coupled to the longitudinal first guide portion and to the longitudinal second guide portion such that the longitudinal second rack is arranged movably with respect to the longitudinal first rack. Longitudinal first rack, longitudinal first guide portion, longitudinal second rack or longitudinal second guide portion are designed to carry an oral retractor.

The connector may comprise at least a first joint rotatably connecting the connector to one of the longitudinal first guide portion and the longitudinal second guide portion such that the longitudinal first rack and the longitudinal second rack may rotate with respect to each other. The first joint may be adapted to connect the connector movably along the one of the longitudinal first guide portion and the longitudinal second guide portion such that the longitudinal first rack and the longitudinal second rack may slidably move with respect to each other. The one of the longitudinal first guide portion and the longitudinal second guide portion may comprise a guide rail on which the connector may slidably move via the first joint.

The connector may comprise a second joint rotatably connecting the connector to the other one of the longitudinal first guide portion and the longitudinal second guide portion such that the longitudinal first rack and the longitudinal second rack may rotate with respect to each other. The second joint may be adapted to connect the connector movably along the other one of the longitudinal first guide portion and longitudinal second guide portion such that the longitudinal first rack and the longitudinal second rack may slidably move with respect to each other. The other one of the longitudinal first guide portion and the longitudinal second guide portion may comprise a guide rail on which the connector may slidably move via the second joint.

The connector may comprise a rod connecting the first joint and the second joint. The first joint and the second joint may comprise a pivot joint, preferably such that the connector may pivot about the one and other one of the longitudinal first guide portion and the longitudinal second guide portion respectively. Further the pivot joints are formed to glide on the guide rails such that the connector may rotate and slide with respect to the longitudinal first guide portion and the longitudinal second guide portion.

The first joint may comprise lock to fix the one of the longitudinal first guide portion and the longitudinal second guide portion such that the connector may not move with respect to the one of the longitudinal first guide portion and the longitudinal second guide portion. The second joint may comprise lock to fix the other one of the longitudinal first guide portion and the longitudinal second guide portion such that the connector may not move with respect to the other one of the longitudinal first guide portion and the longitudinal second guide portion.

The longitudinal first guide portion and the longitudinal second guide portion may have a longitudinal arcuate shape.

The longitudinal second rack may comprise a substantially U-shape or C-shape. The longitudinal second rack may be built to form a frame. The frame may have a substantially U-shape or C-shape when viewed in a side view and another view perpendicular to the side view, e.g. top or front view. The longitudinal second rack may comprise two nested substantially U-shaped or C-shaped rack portions, which are arranged substantially parallel and connected by two distance pieces arranged at respective ends of the two rack portions. One of the two rack portions has a shorter length than the other to fit to the other one in order to be nested and aligned substantially parallel. Thereby, the longitudinal second rack is built as a closed-loop.

The longitudinal second rack may be formed three-dimensionally such that it has a substantially U-shape or C-shape when viewed in a side view, a bottom view and a top view.

The longitudinal first rack may comprise a support portion at a first end portion thereof, wherein the support portion comprises a clamp for connecting to a support structure. The longitudinal first rack may comprise a free end at a second end portion thereof opposite to the first end portion. The support portion may be arranged laterally at a side of a patient such that the frame may be arranged above the face of the patient.

The present invention provides a retractor carrier system for maxillofacial surgery comprising an oral retractor carrier device as described above and a retractor device removably attached to one of the longitudinal first rack and the longitudinal second rack.

The retractor device may comprise at least a first retractor joint rotatably connecting the retractor device to one of the longitudinal first rack and the longitudinal second rack. The first retractor joint may be adapted to connect the retractor device movably along the one of the longitudinal first rack and the longitudinal second rack. The one of the longitudinal first guide portion and the longitudinal second guide portion may comprise a guide rail on which the retractor device may slidably move via the first retractor joint. The first retractor joint may comprise a retractor pivot joint, preferably such that the retractor device may pivot about the one of the longitudinal first rack and the longitudinal second rack. The first retractor joint may further comprise a sliding joint such that the retractor device may slidable move with respect to the first retractor joint. The first retractor joint may comprise respective locks to fix the retractor device and the one of the longitudinal first rack and the longitudinal second rack with respect to each other.

The retractor device may further comprise a second retractor joint rotatably connecting a retractor blade to the retractor device. The second retractor joint may comprise a hinge.

The present invention provides a retractor carrier assembly for maxillofacial surgery comprising a first oral retractor carrier device as described above and a second oral retractor carrier device as described above. The first oral retractor carrier device and the second oral retractor carrier device may be arranged symmetrically such that the longitudinal second rack of the first oral retractor carrier device and the longitudinal second rack of the second oral retractor carrier device are arranged to be aligned to form a common frame. The respective distance pieces may face each other. The corresponding distance pieces of the first oral retractor carrier device and the second oral retractor carrier device may be arranged to be connected or fixed with respect to each other.

The support portion of the first oral retractor carrier device and the support portion of the second oral retractor carrier device may be arranged laterally at opposing sides of a patient such that the common frame may be arranged above the face of the patient.

Other advantages of the present invention are defined in the appended claims.

### BRIEF DESCRIPTION OF THE DRAWINGS

The above described and other issues and features of the present invention will become more apparent from the following detailed description of preferred embodiments of the invention and the accompanying drawings.

FIG. 1 shows an oral retractor carrier device according to a first exemplary preferred embodiment of the present invention in front view when placed on a support structure;

FIG. 2 shows a frame portion of the oral retractor carrier device of FIG. 1;

FIG. 3 shows a connector of the oral retractor carrier device of FIG. 1;

FIG. 4 is a perspective view of a retractor carrier assembly according to a second exemplary preferred embodiment of the present invention;

FIG. 5 shows the retractor carrier assembly of FIG. 4 in front view when placed on a support structure;

FIG. 6 shows a retractor device of the retractor carrier assembly of FIG. 4.

### DETAILED DESCRIPTION OF EXEMPLARY PREFERRED EMBODIMENTS

Preferred embodiments of the present invention are described below with reference to the accompanying drawings, in which the same reference numbers refer to the same features.

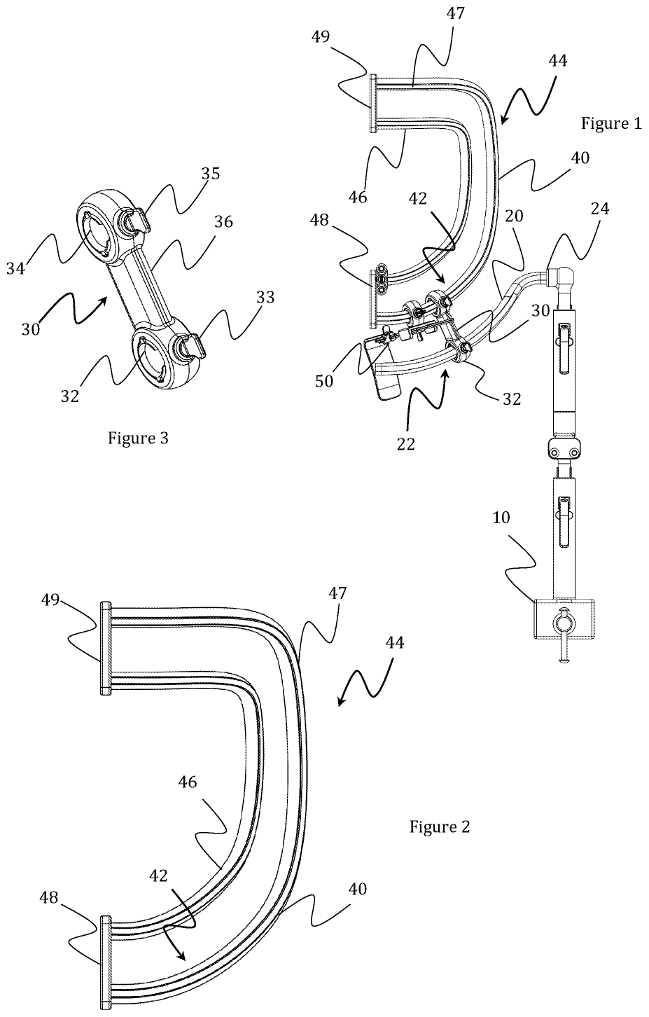

FIG. 1 shows an oral retractor carrier device according to a first exemplary preferred embodiment of the present invention in front view when placed on a support structure 10 such as a surgery table. The oral retractor carrier device comprises a longitudinal first rack 20 and a longitudinal second rack 40 to carry one or more retractor devices 50. The longitudinal first rack 20 has a longitudinal first guide portion 22. The longitudinal second rack 40 has a longitudinal second guide portion 42 that is substantially parallel to the longitudinal first guide portion 22. A connector 30 connects the longitudinal first rack 20 and the longitudinal second rack 40. The connector 30 is attached to the longitudinal first guide portion 22 and the longitudinal second guide portion 42 such that the longitudinal second rack 40 is arranged movably with respect to the longitudinal first rack 20.

As shown in FIGS. 1 and 3, the connector 30 comprises a first joint 32 rotatably connecting the connector 30 to the longitudinal first guide portion 22 such that the longitudinal first rack 20 and the longitudinal second rack 40 are able to rotate about to each other. The first joint 32 is adapted to connect the connector 30 movably along the longitudinal first guide portion 22 and such that the longitudinal second rack 40 may slidably move with respect to the longitudinal first rack 20.

The connector 30 comprises a second joint 34 rotatably connecting the connector 30 to the longitudinal second guide portion 42 such that the longitudinal first rack 20 and the longitudinal second rack 40 is able to rotate about each other. The second joint 34 is adapted to connect the connector 30 movably along the longitudinal second guide portion 42 such that the longitudinal first rack 20 and the longitudinal second rack 40 is able to slidably move with respect to each other. The longitudinal first rack 20 and the longitudinal second rack 40 comprise guide rails on which the connector moves slidably via the first joint 32 and the second joint 34 respectively.

The connector 30 comprises a rod 36 connecting the first joint 32 and the second joint 34. The first joint 32 and the second joint 34 comprise pivot joints such that the connector 30 is able to pivot about the longitudinal first guide portion 22 and the longitudinal second guide portion 42. Further the pivot joints are formed to glide on the guide rails such that the connector may rotate and slide with respect to the longitudinal first guide portion 22 and the longitudinal second guide portion 42.

The first joint 32 comprises a first lock 33 to fix the longitudinal first guide portion 22 such that the connector 30 cannot move with respect to the longitudinal first guide portion 22. The second joint 34 comprises a second lock 35 to fix the longitudinal second guide portion 42 such that the connector 30 cannot move with respect to the longitudinal second guide portion 42.

The longitudinal first guide portion 22 and the longitudinal second guide portion 42 have both matching longitudinal arcuate shapes.

As shown in FIGS. 1 and 2, the longitudinal second rack 40 comprises a substantially U-shape or C-shape. The longitudinal second rack 40 forms a frame 44. The frame 44 has a substantially U-shape or C-shape when viewed in a side view and another view perpendicular to the side view, e.g. top or front view. The longitudinal second rack 40 comprises two substantially U-shaped or C-shaped rack portions 46, 47, which are arranged substantially parallel and connected by two distance pieces 48, 49 arranged at respective ends of the two rack portions 46, 47. One 46 of the two rack portions has a shorter length than the other 47 to fit to the other one 47 in order to be aligned substantially parallel.

The longitudinal second rack 40 is formed three-dimensionally such that it has a substantially U-shape or C-shape when viewed in a side view, a bottom view and a top view.

The longitudinal first rack 20 comprises a support portion 24 at a first end portion thereof, wherein the support portion 24 comprises a clamp for connecting to a support structure 10. The longitudinal first rack 20 may comprise a free end at a second end portion thereof opposite to the first end portion.

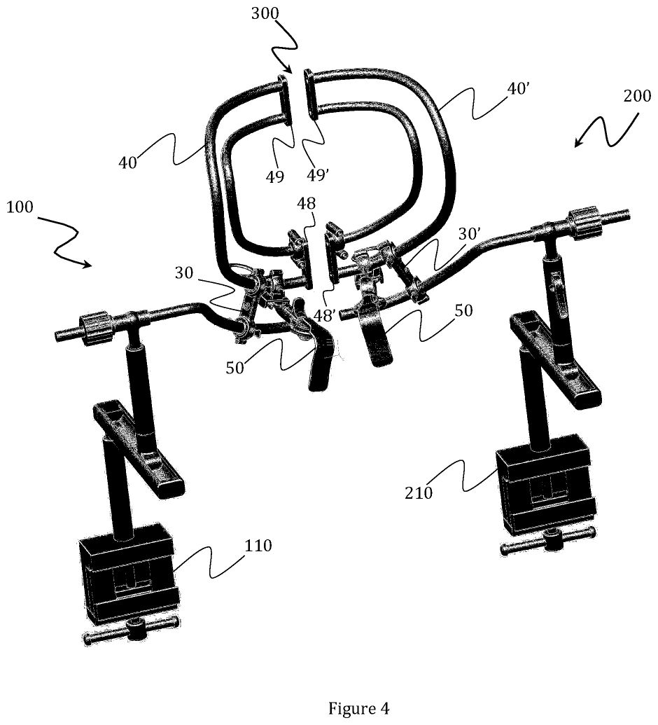

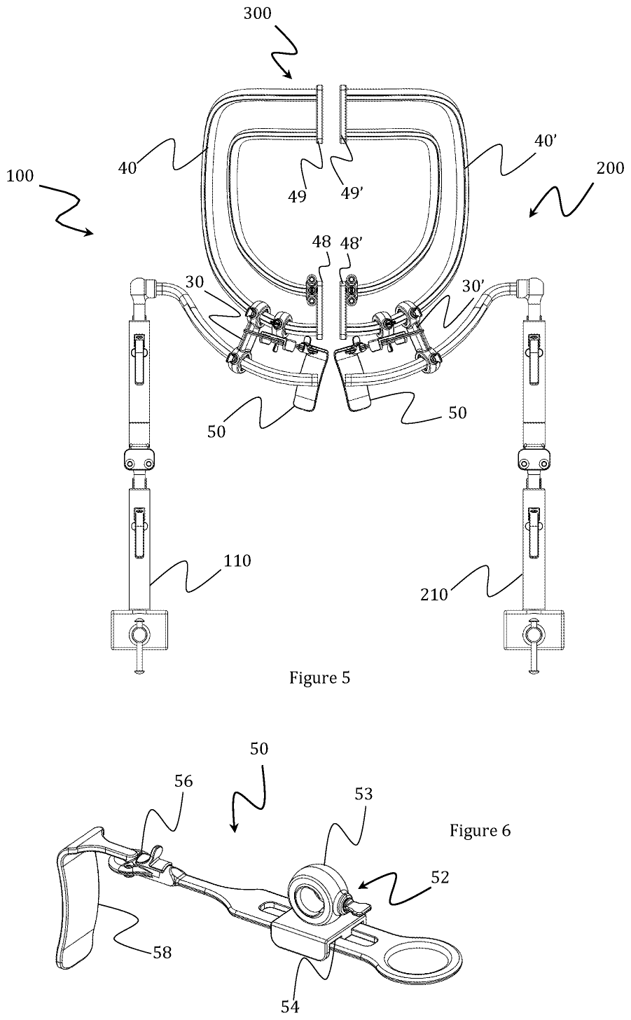

FIG. 1 as well as FIGS. 4 and 5 show a retractor carrier system for maxillofacial surgery comprising an oral retractor carrier device and a retractor device 50 removably attached to the longitudinal first rack 20.

As shown in FIG. 6, the retractor device 50 comprises a first retractor joint 52 rotatably connecting the retractor device 50 to the longitudinal second rack 40 (as shown in FIG. 1 as well as FIGS. 4 and 5). The first retractor joint 52 is adapted to connect the retractor device 50 movably along the longitudinal second rack 40. The longitudinal first guide portion 22 and the longitudinal second guide portion 42 comprise guide rails on which the retractor device 50 slidably moves via the first retractor joint 52. The first retractor joint 52 comprises a retractor pivot joint 53, such that the retractor device 50 is able to pivot about the the longitudinal second rack 40. The first retractor joint 52 further comprises a sliding joint 54 such that the retractor device 50 moves slidably on the first retractor joint 52. The first retractor joint 52 comprises respective locks to fix the retractor device 50 and the longitudinal second rack 40 with respect to each other.

The retractor device 50 further comprises a second retractor joint 56 rotatably connecting a retractor blade 58 to the retractor device 50. The second retractor joint 56 comprises a hinge.

FIGS. 4 and 5 show a retractor carrier assembly for maxillofacial surgery comprising a first oral retractor carrier device 100 and a second oral retractor carrier device 200. The first oral retractor carrier device 100 and the second oral retractor carrier device 200 (both corresponding to the oral retractor carrier device shown in FIG. 1) are arranged symmetrically such that the longitudinal second rack 40 of the first oral retractor carrier device 100 and the longitudinal second rack 40′ of the second oral retractor carrier device 200 are aligned to form a common frame 300. The respective distance pieces 48, 48′, 49, 49′ face each other. The corresponding distance pieces 48, 48′, 49, 49′ of the first oral retractor carrier device 100 and the second oral retractor carrier device 200 are fixed with respect to each other via a first support structure 110 and a second support structure 210 respectively. Alternatively or additionally, the fixing may be performed by linking or attaching the corresponding distance pieces 48, 48′, 49, 49′.

The above description is not limiting. Many other examples or adaptations of the above embodiments will be apparent to those skilled in the art upon review of the above description without departing from the scope of the invention. Each of the following claims is incorporated herein as a separate preferred embodiment. Certain features of the disclosed embodiments are shown at an exaggerated scale or schematically, and some details of conventional elements are omitted for clarity and brevity.

The word “a” in this specification appropriately means “one or more” or “at least one”. The term “based on” can mean “using at least a part of . . . or a derivation of . . . ”. The word “first” can mean “initial”, “current” or “provisional” and vice versa. The word “subsequent” can mean “next” or “following” and vice versa. The word “previous” can mean “prior” and vice versa. An algorithm according to the invention includes methods for the step-by-step transformation of character rows, including calculation processes according to a specific, possibly repetitive scheme. Instead of the camera, the imaging device may comprise an infrared or thermal imaging device, an ultrasonic imaging device or a magnetic imaging device having a respective sensor adapted to produce a corresponding image of the object.

The preferred embodiments of the invention have been described in the drawings and the description of the preferred embodiments. While these disclosures directly describe the above embodiments, it is understood that those skilled in the art may consider modifications and/or variations to the specific embodiments shown and described herein.

1. An oral retractor carrier device, comprising:
a longitudinal first rack having a longitudinal first guide portion;
a longitudinal second rack having a longitudinal second guide portion,
wherein the longitudinal first guide portion and the longitudinal second guide portion are aligned substantially parallel and coupled to each other such that the longitudinal second rack is arranged movably with respect to the longitudinal first rack.

2. The oral retractor carrier device according to claim 1, further comprising at least a connector coupled to the longitudinal first guide portion and to the longitudinal second guide portion such that the longitudinal second rack is arranged movably with respect to the longitudinal first rack.

3. The oral retractor carrier device according to claim 1, wherein the longitudinal first guide portion and the longitudinal second guide portion comprise guide rails.

4. The oral retractor carrier device according to claim 1, wherein the connector comprises at least a first joint rotatably connecting the connector to one of the longitudinal first guide portion and the longitudinal second guide portion such that the longitudinal first rack and the longitudinal second rack may rotate with respect to each other, wherein the pivot joints are formed to glide on the guide rails such that the connector may rotate and slide with respect to the longitudinal first guide portion and the longitudinal second guide portion.

5. The oral retractor carrier device according to claim 1, wherein the connector comprises a second joint rotatably connecting the connector to the other one of the longitudinal first guide portion and the longitudinal second guide portion such that the longitudinal first rack and the longitudinal second rack may rotate with respect to each other.

6. The oral retractor carrier device according to claim 1, wherein the first joint and the second joint comprise a pivot joint, such that the connector is able to pivot about the one and other one of the longitudinal first guide portion and the longitudinal second guide portion respectively.

7. The oral retractor carrier device according to claim 1, wherein the longitudinal first guide portion and the longitudinal second guide portion have a longitudinal arcuate shape.

8. The oral retractor carrier device according to claim 1, wherein the longitudinal second rack comprises a substantially U-shape or C-shape.

9. The oral retractor carrier device according to claim 1, wherein the longitudinal second rack is built to form a frame having a substantially U-shape or C-shape when viewed in a side view and another view perpendicular to the side view.

10. The oral retractor carrier device according to claim 1, wherein the frame comprises two matching substantially U-shaped or C-shaped rack portions, which are arranged substantially parallel and connected by two distance pieces arranged at respective ends of the two rack portions.

11. A retractor carrier system for maxillofacial surgery comprising an oral retractor carrier device according to claim 1 and a retractor device removably attached to one of the longitudinal first rack and the longitudinal second rack.

12. The retractor carrier system according to claim 11, wherein retractor device comprises at least a first retractor joint rotatably connecting the retractor device to one of the longitudinal first rack and the longitudinal second rack.

13. The retractor carrier system according to claim 11, wherein first retractor joint further comprises a sliding joint such that the retractor device is able to slidable move with respect to the first retractor joint.

14. The retractor carrier system according to claim 11, wherein retractor device further comprises a second retractor joint rotatably connecting a retractor blade to the retractor device.

15. A retractor carrier assembly for maxillofacial surgery comprising a first oral retractor carrier device according to claim 1 and a second oral retractor carrier device according to claim 1.

16. The retractor carrier assembly according to claim 15, wherein the first oral retractor carrier device and the second oral retractor carrier device are arranged symmetrically side by side such that the longitudinal second rack of the first oral retractor carrier device and the longitudinal second rack of the second oral retractor carrier device are aligned to form a common frame.

17. The retractor carrier assembly according to claim 15, wherein the respective distance pieces face each other and are fixed with respect to each other via support structures connected to the longitudinal first rack of the first oral retractor carrier device and the longitudinal first rack of the second oral retractor carrier device.
